(19) United States
(12) Patent Application Publication
(10) Pub. No.: US 2011/0178477 A1
(43) Pub. Date: Jul. 21, 2011

(54) TROCAR DEVICE FOR PASSING A SURGICAL TOOL

(76) Inventors: Guillaume Morel, Maisoncelles en Brie (FR); Nabil Zemiti, Fontenay Aux Roses (FR)

(21) Appl. No.: 10/585,412

(22) PCT Filed: Jan. 7, 2005
(86) PCT No.: PCT/FR2005/000042

§ 371 Date: Jun. 22, 2009

(30) Foreign Application Priority Data
Jan. 7, 2004 (EP) 04290028.2

(51) Int. Cl. A61B 17/34; A61B 19/00

(52) U.S. Cl. 604/264

(57) ABSTRACT

This invention relates to a trocar device (9) for passing a surgical instrument (15), characterised in that it includes measuring means (10, 17, 19) for measuring the force exerted by the said instrument (15) on the internal organs of a patient (6).

Advantageously, the measuring means take the form of at least one force transducer (10, 19) arranged on the said trocar (9).

This invention relates to a trocar device for passing a surgical instrument.

Operating laparoscopy consists in performing surgical procedures with a miniaturised surgical instrument with a small diameter that makes it possible to pass it through a trocar, which is a hollow tube inserted through the abdominal or thoracic wall of a patient.

More specifically, laparoscopy consists in inserting (a) a laparoscope into the abdominal or thoracic wall of a patient to enable the surgeon to see and examine, and (b) instruments for performing a procedure under visual inspection via the laparoscope, without having to open the entire abdomen.

Even though a laparoscopy operation can be performed entirely by hand, it is sometimes performed by means of a robotised system.

In that case, in order to make the laparoscopy more accurate, the surgeon does not directly manipulate the surgical tools, but does so through an electrical-mechanical interface.

In that way, the surgeon moves control arms through an interface, to control robotised arms that work directly on the patient, where the robotised arms are connected to surgical tools or a laparoscope, for instance.

However, a problem found while using these robotised systems is that the surgeon cannot directly estimate the force applied by the laparoscope or the instruments on the internal organs of the patient.

Consequently, the surgeon has to make up for the loss of feeling by a visual estimation of the deformation of the organs as it is seen on the screen displaying the laparoscopic image.

That is particularly a problem in the case of endosurgical operations that call for very precise micro-surgical movements, where all the measurement parameters need to be known.

At the current time, for conventional applications (non endoscopic), there are remotely operated control systems that enable the surgeon to control the force applied by the operator on the patient.

However, these methods are based on the hypothesis that the interaction that needs to be felt can be measured or estimated.

That is difficult to envisage in endoscopic surgery, since in that case, force transducers that meet sterility, dimensional, accuracy and cost requirements would need to be installed inside the patient.

It would be particularly advantageous to have an accurate estimate of the force of interaction between instrument and internal organ, without using an internal transducer.

The object of this invention is to solve that problem with a simple, cheap and reliable instrumental device that can be installed on existing robotised remotely operated systems.

This invention relates to a trocar device for passing a surgical instrument, characterised in that it has means to measure the force applied by the said instrument on the internal organs of a patient, the said measurement means taking the form of at least one force transducer fitted on the trocar, the force transducer being advantageously formed as a roller with a central orifice, and placed between the trocar and a guide.

The guide advantageously takes the form of a tubular element with a lengthwise axis (X-X) having a circular plate perpendicular to (X-X) at one of its ends and is inserted in the said central orifice of the said force transducer and the said trocar device.

According to a first embodiment of the trocar device according to the invention, the instrument is moved by a robotised arm and a second force transducer is placed between the end of the robotised arm and the surgical instrument.

According to a second embodiment, the instrument is moved by a displacement mechanism placed on the guide, preferably by a roller type displacement mechanism, and the trocar device is moved by the end of a robotised arm.

Advantageously, the movement of the robotised arm is generally controlled from an interface.

This invention is now described using examples that are purely illustrative and do not limit the scope of the invention, with reference to the accompanying drawings, wherein:

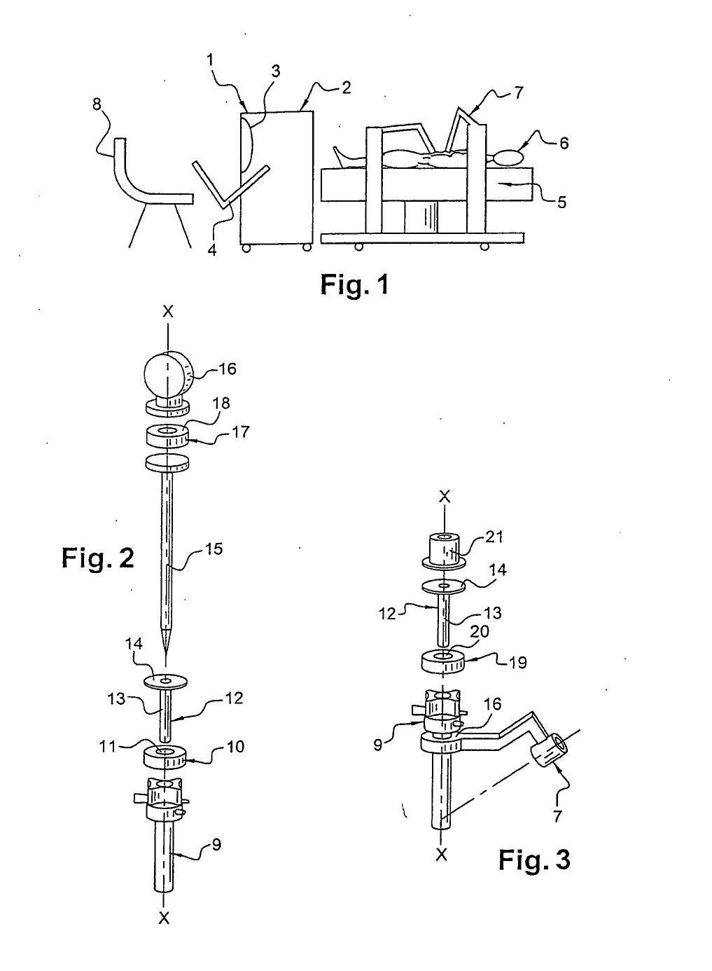

FIG. 1 shows a schematic view of a remotely operated endosurgical manipulation system,

FIG. 2 is an exploded perspective view of a trocar device according to the invention where the surgical instrument is moved by a robotised arm, and

FIG. 3 is an exploded perspective view of a trocar device according to the invention, where the surgical instrument is moved by a displacement mechanism.

This invention is described for use during a surgical operation of the laparoscopic type, it being understood that the general principle of the invention may be applied among others to all types of remotely operated surgical operation where a trocar is used or to any system for introducing trainee surgeons to surgical operations or training them in such is operations.

FIG. 1 shows a robotised system 1 used to perform a remotely operated surgical procedure from interface 2, and more specifically for endosurgical operations.

Interface 2 takes the form of a display screen 3 and a pair of control arms 4 which can be manipulated by a surgeon.

Interface 2 is used along with an operating table 5 on which the patient 6 undergoing the operation is placed.

Operating table 5 is used along with a set of robotised arms 7, it being understood that a robotised arm may be used along with a laparoscope, a camera, a set of forceps, a scalpel etc.

Advantageously, the displacement of the two control arms 4 by the surgeon leads to the displacement of robotised arms 7, it being understood that several robotised arms 7 may be controlled by the two control arms 4, interface 2 making it possible to select the robotised arms 7 that the surgeon wishes to guide remotely.

Advantageously, interface 2 has a seat 8 to offer greater comfort to the surgeon during the operation and reduce fatigue due to prolonged standing during the procedure.

FIG. 2 is an exploded perspective view of a trocar device used along with an instrument moved by a robotised arm.

Advantageously, a trocar 9 known in the art is used, i.e. it takes the form of a hollow tubular element and is inserted in the abdominal wall of a patient 6 during the surgical procedure.

Trocar 9 is fitted with a first force transducer 10 that is known in the art and is commercially available, e.g. the transducer known as ATI Nano43 (registered trademark).

The first force transducer 10 is cylindrical in shape, preferably in the form of a roller, and has a central orifice 11 in which a guide 12 that is passive and sealed during displacement can be inserted.

Guide 12 takes the form of a hollow tubular element 13 with, at one end, a circular plate 14 arranged transversal to the lengthwise axis (X-X) of tubular element 13.

Advantageously, tubular element 13 is inserted in the central orifice 11 of the first force transducer and in trocar 9.

Guide 12 is advantageously made of sterilisable material, such as stainless steel.

In order to make the assembly made up of guide 12 and first force transducer 10 tight, a rubber seal that is known in the art is added between the two elements (not shown in the figure but known in the art).

An instrument 15, for example a laparoscope, connected to end 16 of a robotised arm 7 is slideable in guide 12 with one or two degrees of freedom, i.e. displaced in relation to (X-X) and/or rotated around (X-X).

It is understood that instrument 15 is any type of surgical instrument known in the art and able to be inserted in a trocar 9.

A second force transducer 17, known in the art and commonly available commercially, for instance a transducer known as ATI Nano43 (registered trademark), is arranged between the end 16 of a robotised arm 7 and the instrument 15.

The choice of the form and functions of the second force transducer 17 is independent of the choice of the form and function of first force transducer 11.

Advantageously, the second transducer 17 is cylindrical in shape, for example in the form of a roller comprising a central orifice 18.

In order to know the interaction force between the instrument 15 and the internal organs of the patient 6, an estimator has been developed on the basis of dynamic equations that take account of the forces and moments of torsion at the connection between the trocar 9 and the instrument 15.

More specifically, by expressing the torsor, i.e. the force and moment at an arbitrary point of the mechanical action applied by body i on body j as W_{i→j}, and the torsor representing the action of the gravitational field on body i as W_{gravity→i}, the trocar can be modelled statically, assuming the system is in equilibrium.

By leaving out dynamic effects, the equation of equilibrium of instrument 15 can be determined as follows:

ΣW_{exterior→instrument}=0=W_{second}_{_{—}}_{force}_{_{—}}_{transducer→instrument}+W_{guide→instrument}+W_{organ→instrument}+W_{gravity→instrument}

However, in order to take account of dynamic effects, transducers may be arranged to measure or estimate the acceleration of bodies and use measurements jointly with an object model to make up for the inertial effects, as this technique is well known to the one skilled in the art.

The equation of equilibrium of guide 12 is then determined as follows:

ΣW_{exterior→guide}=0=W_{instrument→guide}+W_{first}_{_{—}}_{force}_{_{—}}_{transducer→guide}+W_{gravity→guide}

The first force transducer 10 can measure W_{first}_{_{—}}_{transducer→guide} and the second force transducer 17 can measure W_{second}_{_{—}}_{transducer→instrument}.

On the basis of these two equations above, the interaction force of instrument 15 and the internal organs of the patient 6 can be determined.

The equation obtained is:

W_{instrument→organ}=W_{first}_{_{—}}_{transducer→guide}+W_{second}_{_{—}}_{transducer→instrument}+W_{gravity}

Where W_{gravity}=W_{gravity→guide}+W_{gravity→instrument}

Once W_{first}_{_{—}}_{transducer→guide} and W_{second}_{_{—}}_{transducer→instrument} have been measured, W_{first}_{_{—}}_{transducer→guide} is expressed in the same base and at the same point as measurement W_{second}_{_{—}}_{transducer→instrument} the implementation of that estimation being obvious for one skilled in the art.

The gravity force torsor will be calculated thereafter as follows

Ŵ_{gravity}=Ŵ_{gravity→instrument}+Ŵ_{gravity→guide}

That calculation, which based on a weight model, is obvious for one skilled in the art.

Finally, when all the torsors are expressed in the measurement base W_{second}_{_{—}}_{transducer→instrument} at the measurement point of W_{second}_{_{—}}_{transducer→instrument}, the interaction of instrument 15 on the internal organs of patient 6 is then estimated, that is:

Ŵ_{instrument→organ}=W_{second}_{_{—}}_{force}_{_{—}}_{transducer→instrument}+W_{first}_{_{—}}_{force}_{_{—}}_{transducer→guide}+Ŵ_{gravity}

That estimation is accomplished by a calculator known in the art, and is used to display the force applied by the instrument on the internal organs on interface 2 with the help of electrical means of a type known in the art.

Also, physical parameters such as masses and the centre of gravity and geometric parameters such as the position and relative direction of force transducers, the position of instrument 15 relative to trocar 9, are either known a priori if a model has been identified or are taken from an initial calibration procedure, the implementation of which is conventional for one skilled in the art.

FIG. 3 is an exploded view of a trocar used along with a force transducer and a displacement mechanism.

FIG. 3 is an alternative representation of the trocar device according to the invention, where it is only necessary to incorporate a single force transducer to determine the forces of interaction between a surgical is instrument and the internal organs of a patient.

In the description below, the same elements of reference as those in FIG. 2 will have the same reference numbers.

In order to appreciate the force exerted by an instrument 15 on the internal organs of a patient 6, a guide 12 in the form of a tubular element 13 and a circular plate 14 is placed on a trocar 9 known in the art.

Advantageously, guide 12 takes the form of a tubular element 13 having, on one of its ends, a circular plate 14 perpendicular to the lengthwise axis (X-X) of the tubular element 13.

Between guide 12 and trocar 9 is arranged a force transducer 19 of the same type as those used previously for the trocar of FIG. 2, i.e. in the form of a roller with a central orifice 20 for passing instrument 15 and passive guide 12.

Thus, force transducer 19 is known in the art and is commonly commercially available, such as a transducer known as ATI Nano43 (registered trademark).

The tubular element 13 of guide 12 is inserted in the central orifice 20 of force transducer 19 and in trocar 9.

A displacement mechanism 21 is placed on circular plate 14 of guide 12 and is such that it can enable the lengthwise displacement along (X-X) of an instrument 15 (not shown in FIG. 3 for more clarity, but of the same type as that in FIG. 2).

Advantageously, Ie displacement mechanism 21 is known in the art, for example a roller displacement mechanism.

Trocar 9 is directly set in motion by the end 16 of robotised arm 7.

Alternatively, trocar 9 may be set in motion by an independent robotised system that can tilt trocar 9 in different directions.

Therefore, any force between the instrument 15 and the internal organs of the patient 6 is transmitted by the displacement mechanism 21 to force transducer 19.

Advantageously, a force feedback control known in the art by the one skilled in the art has been developed to make it possible, with an external transducer 19, to control the forces within the body in spite of the friction produced by trocar 9.

More precisely, as with the trocar in FIG. 2, for the estimation of the force of interaction between instrument 15 and internal organs 6, the torsor, i.e. the force and moment at a random point of the mechanical action exerted by body i on body j, is expressed as W_{i→j}, and the torsor representing the action of the field of gravity on body i is expressed as W_{gravity→i} so that the trocar can be modelled statically, assuming that the system is in equilibrium.

That is because at the speeds used in surgery, the inertial effects of accelerations may be considered to be negligible.

In order to model and estimate the various forces of trocar 9, the equilibrium equations of instrument 15, displacement mechanism 21 and is guide 12 are determined as follows:
Equilibrium equation of instrument 15:

ΣW_{exterior→instrument}=0=W_{displacement}_{_{—}}_{mechanism→instrument}+W_{guide→instrument}+W_{organ→instrument}+W_{gravity→instrument}
Equilibrium equation of displacement mechanism 21:

ΣW_{exterior→displacement}_{_{—}}_{mechanism}=0=W_{instrument→displacement}_{_{—}}_{mechanism}+W_{guide→displacement}_{_{—}}_{mechanism}+W_{gravity→displacem}
Equilibrium equation of guide 12:

ΣW_{exterior→guide}=0=W_{displacement}_{_{—}}_{mechanism→guide}+W_{instrument→guide}+W_{force}_{_{—}}_{transducer→guide}+W_{graviy→guide}

It should be noted that W_{force}_{_{—}}_{transducer→guide} is the force measured by force transducer 19.

The interaction force between instrument 15 and the organs of the patient 16 is to be estimated, i.e. W_{organ→instrument}.

By combining the three previous equations, the following equation is obtained:

W_{force}_{_{—}}_{transducer→guide}=−W_{displacement}_{_{—}}_{mechanism→guide}−W_{instrument→guide}−W_{gravity→guide}

W_{force}_{_{—}}_{transducer→guide}=W_{guide→displacement}_{_{—}}_{mechanism}+W_{guide→instrument}−W_{gravity→guide}

But:

W_{guide→displacement}_{_{—}}_{mechanism}=−W_{instrument→displacement}_{_{—}}_{mechanism}−W_{gravity→displacement}_{_{—}}_{mechanism}

and

W_{guide→instrument}=−W_{displacement}_{_{—}}_{mechanism→instrument}−W_{organ→instrument}−W_{gravity→instrument}

Which finally provides:

W_{force}_{_{—}}_{transducer→guide}=W_{instrument→organ}−(W_{gravity→displacement}_{_{—}}_{mechanism}+W_{gravity→instrument}+W_{gravity→guide})

Therefore, the force measured by transducer 19 is the internal force between instrument 15 and the internal organs of patient 6, except for the weight of the assembly made up of instrument 15, passive guide 12 and displacement mechanism 21.

Besides, it should be noted that the friction between passive guide 12 and instrument 15 and the interaction between the abdominal wall and trocar 9 is not involved in the measurement.

Therefore, to estimate the interaction between instrument 15 and the internal organs of the patient 6, the torsor delivered by force transducer 19, i.e. W_{force}_{_{—}}_{transducer→guide}, must be measured first.

Thereafter, it is necessary to calculate the torsor of the force of gravity, i.e.:

Ŵ_{gravity}=W_{gravity→displacement}_{_{—}}_{mechanism}+W_{gravity→instrument}+W_{gravity→guide}

Now it is possible to estimate the interaction between instrument 15 and the internal organs of patient 6 with equation:

Ŵ_{instrument→organ}=W_{force}_{_{—}}_{transducer→guide}+Ŵ_{gravity}

To calculate the gravity force torsor, several methods are commonly used:
Either the weight model (mass and location of the centre of gravity) of instrument 15, displacement mechanism 21 and guide 12 is perfectly known.
In that case, the gravity torsor is calculated on the basis of the measurement of the direction of trocar 9, taken by position sensors arranged on robotised arm 16 directly connected to trocar 9, and from the measurement of the position of instrument 15 in relation to guide 12, taken by position sensors placed on displacement mechanism 21, this measurement method being obvious for one skilled in the art.
Or one or more parameters required for the calculation at the basis of the model are not known.
In that case, the operation is preceded by calibration. To that end, the system is placed in different geometrical configurations with the help of displacement mechanism 21 and end 16 of robotised arm 7, while ensuring that instrument 15 is not in contact with the internal organs of the patient 6.
It is now possible to either build a correspondence table or to identify the parameters of the weight model according to an operating procedure that is well known to one skilled in the art.

It is also possible to express the torsor of the force exerted by instrument 15 on the internal organs of the patient 6 in a base related to instrument 15, instead of force transducer 19, and at a point corresponding to the end of instrument 15, instead of a point relating to force transducer 19. In that case, it suffices to know the relative position of the instrument in relation to transducer 19, which can be calculated according to methods that are conventional for one skilled in the art.

In that way, it is possible to determine the forces of interaction between a surgical instrument 15 and an internal organ of a patient 6 from force transducers (10, 17, 19) arranged outside trocar 9.

The estimation of the interaction force between the surgical instrument 15 and the internal organs of a patient 6 is achieved on the basis of the torsors measured by the force transducers (10, 17, 19), a calculator known in the art being used for the instantaneous display of the force exerted by instrument 15 on the internal organs of patient 6 on interface 2.

Advantageously, the surgeon can, from interface 2, determine the maximum force that is to be applied on the internal organs of the patient 6, which cannot be exceeded.

That limitation of the force applied to internal organs 6 is used to ensure that a strong uncontrolled movement of a higher force does not affect the internal organs of the patient 6.

Advantageously, interface 2 has means to monitor the force applied by the instrument and/or means to restore the force exerted by the instrument to the surgeon by means of control arms 4.

1. Trocar device (9) for passing a surgical instrument (15), characterised in that it includes measuring means (10, 17, 19) for measuring the force exerted by the said instrument (15) on the internal organs of a patient (6).

2. Device according to claim 1, characterised in that the said measuring means take the form of at least one force transducer (10, 19).

3. Device according to claim 2, characterised in that the said force transducer (10, 19) is arranged on the said trocar (9).

4. Device according to claim 2, characterised in that the said transducer (10, 19) takes the form of a roller with a central orifice (11, 20).

5. Device according to claim 2, characterised in that the said force transducer (10, 19) is arranged between the said trocar (9) and a guide (12).

6. Device according to the preceding claim, characterised in that the said guide (12) takes the form of a tubular element (13) with a lengthwise axis (X-X) having a circular plate (14), perpendicular to (X-X), at one of its ends.

7. Device according to claim 5, characterised in that the said guide (12) is inserted in the said central orifice (11, 20) of the said force transducer (10, 19) and in the said trocar device (9).

8. Device according to claim 1, characterised in that the said instrument (15) is set in motion by a robotised arm (7).

9. Device according to claim 8, characterised in that a second force transducer (17) is arranged between the end (16) of the said robotised arm (7) and the said surgical instrument (15).

10. Device according to claim 1, characterised in that the said instrument (15) is set in motion by a displacement mechanism (21).

11. Device according to the preceding claim, characterised in that the said displacement mechanism (21) is arranged on the said guide (12).

12. Device according to claim 10, characterised in that the said displacement mechanism (21) is a roller type displacement mechanism.

13. Device according to claim 10, characterised in that it is set in motion by the end (16) of a robotised arm (7).

14. Device according to 8, characterised in that the displacement of the said robotised arm (7) is controlled from an interface (2).
